(19) United States
(12) Patent Application Publication
(10) Pub. No.: US 2013/0195996 A1
(43) Pub. Date: Aug. 1, 2013

(54) METHOD OF PRODUCING PLEURODESIS

(71) Applicants: Anthony Looper, Lake Zurich, IL (US); Griffin Strole, Chicago, IL (US)

(72) Inventors: Anthony Looper, Lake Zurich, IL (US); Griffin Strole, Chicago, IL (US)

(73) Assignee: CAREFUSION 2200, INC., San Diego, CA (US)

(21) Appl. No.: 13/696,482

(22) PCT Filed: May 5, 2011
(86) PCT No.: PCT/US11/35304

§ 371 Date: Apr. 16, 2013

Related U.S. Application Data
(60) Provisional application No. 61/332,537, filed on May 7, 2010.

(51) Int. Cl. A61K 33/38; A61M 27/00

(52) U.S. Cl. CPC A61K 33/38; A61M 27/00
USPC 424/618; 604/540

(57) ABSTRACT

The present invention provides methods of producing pleurodesis in a mammalian subject, comprising administration of a low dosage of a sclerosing agent such as silver or a salt of silver.

### FIELD OF THE INVENTION

The present invention provides methods of producing pleurodesis in a mammalian subject, comprising administration of a low dosage of a sclerosing agent such as saver or a salt of silver.

### BACKGROUND OF THE INVENTION

The pleural cavity and the pleura serve an important function of aiding in the optimal functioning of the lungs during respiration. Diseases affecting the pleural cavity and pleura include pleural effusions and pneumothorax. Pleural effusions involve the build-up of fluid around the lungs. Pleural effusions can be associated with conditions such as cancer, tuberculosis, congestive heart failure, pneumonia, pulmonary emboli, viral disease, cirrhosis, post coronary artery bypass graft surgery, gastrointestinal disease, tuberculosis, and mesothelioma. Pneumothorax occurs when air or gas is present in the pleural cavity.

Patients with pleural diseases such as symptomatic pleural effusions or pneumothorax are typically treated with thoracentesis to remove fluid or air, and/or chemical or mechanical pleurodesis. Pleurodesis involves irritation of the parietal and/or visceral layers of the pleura in order to close off the pleural space and prevent further fluid and/or air accumulations. Pleurodesis is typically characterized by the creation of fibrous adhesions between the parietal and visceral layers of the pleura. Chemical pleurodesis can be achieved with the insertion of sclerosing agents, typically by catheter, into the pleural space. Sclerosing agents include talc, tetracycline, doxycycline, minocycline, doxorubicin, povidone iodine, bleomycin, and silver nitrate.

The degree of pleurodesis can be measured by a Pleurodesis Score. The Pleurodesis Score is typically measured on a scale of 1 to 8, wherein 1 represents no adhesions and 8 represents many adhesions between the visceral and parietal pleura with symphysis involving greater than 50% of the hemithorax.

Silver nitrate has been used as a sclerosing agent to achieve pleurodesis. However, widespread use has been abandoned since the late 1980s due to severe pain, fever, and relatively large amount of effusion associated with an injection of silver nitrate into the pleural space. Known methods involved the use of high dosages of silver nitrate, such as a 10% concentration (typically 200 mg AgNO₃) in a single dosage. There is a need in the art for a method of treating or preventing recurrent pleural effusions by administration of a sclerosing agent which is effective in achieving pleurodesis while decreasing the adverse effect of pain associated with known methods.

Marchi et al. (“Intrapleural low-dosage silver nitrate elicits more pleural inflammation and less systemic inflammation than low-dosage talc,” Chest, 2005; 128:1798-1804) discloses the induction of a relatively intense pleural inflammation in rabbits after a single dosage administration of 0.1% silver nitrate.

Marchi et al. (“Low doses of silver nitrate induce pleurodesis with a limited systemic response,” Respirology, 2009, Vol. 14, pp. 885-89) discloses intrapleural injection of 0.1% silver nitrate at 0 hours, 24 hours, and 48 hours in rabbits. Marchi et al. also discloses administration of a single injection of 0.5% silver nitrate or 400 mg/kg of talc in rabbits.

Texeira et al. (“Low concentration silver nitrate pleurodesis in rabbits: optimal concentration for rapid and complete sclerosing effect,” Lung, 2003; 181: 353-359) discloses significant pleurodesis in rabbits after intrapleural injection of 0.5% silver nitrate and no sclerosing effects after a single intrapleural injection of 0.25% silver nitrate.

Vargas et al. (“Experimental pleurodesis in rabbits induced by silver nitrate or talc: 1-year follow-up,” Chest, 2001; 119: 1516-1520) discloses a relatively superior pleurodesis result in rabbits after administration of 2 mL of 0.25% silver nitrate. This result also persists for at least a year.

Vargas et al. (“Effectiveness of silver nitrate compared to talc slurry as pleural sclerosing agent in rabbits. influence of concomitant intrapleural lidocaine,” Rev. Hosp. Clin. Fac. Med. S. Paulo, 1999; 54(6): 199-208), Vargas et al. (“Lung damage in experimental pleurodesis induced by silver nitrate or talc,” Chest, 2002; 122: 2122-2126), and Vargas et al. (“Comparison of silver nitrate and tetracycline as pleural sclerosing agents in rabbits,” Chest, 1995; 108: 1080-1083) disclose the injection of 0.5% silver nitrate in rabbits to achieve pleurodesis.

Andersen et al. (“Results of silver nitrate pleurodesis in spontaneous pneumothorax,” Dis. Chest, September 1968, Vol. 54, No. 3, pp. 230-233) discloses treating spontaneous pneumothorax in humans by administering 2 mL of a 10% silver nitrate solution through a thoracoscope.

Stowe et al. (“Open thoracotomy for pneumothorax in cystic fibrosis,” American Review of Respiratory Disease, 1975, Vol. 111, pp. 611-617) discloses administration of 2 to 10 mL of a 1% silver nitrate solution, injected as a single bolus, in human patients with cystic fibrosis.

Schuster et al. (“Management of pneumothorax in cystic fibrosis,” Journal of Pediatric Surgery, 1983, Vol. 18, No. 4, pp. 492-497) discloses the administration of silver nitrate 1% as a single 10 mL instillation into the pleural space of human patients with cystic fibrosis.

Wied et al. (“Tetracycline versus silver nitrate pleurodesis in spontaneous pneumothorax,” J Thorac Cardiovasc Surg, 1983, Vol. 86, pp. 591-593) discloses producing pleurodesis in a human subjects comprising administration of 2 mL of a 10% silver nitrate solution evenly over the surface of the lung through a ureteral catheter.

Musani et al. (“Outpatient management of malignant pleural effusions with small-bore, tunneled pleural catheters,” Respiration, 2004, Vol. 71, pp. 559-566) discloses a retrospective analysis of patients undergoing outpatient pleural catheter placement for recurrent symptomatic malignant pleural effusion.

Tremblay et al. (“Single-center experience with 250 tunneled pleural catheter insertions for malignant pleural effusion,” Chest, 2006, Vol. 129, pp. 362-368) discloses a study of two hundred and fifty tunneled pleural catheter procedures for malignant pleural effusions in human subjects. The catheters were kept in place for a median duration of 58 days.

Warren et al. (“Identification of clinical factors predicting Pleurx® catheter removal in patients treated for malignant pleural effusion,” Eur J Cardiothorac Surg, 2008; Vol. 33, pp. 89-94), discloses a study of patients undergoing insertion of a Pleurx® catheter in the management of malignant pleural effusions. Warren et al. discloses that spontaneous pleural symphysis in some patients.

Putnam et al. (“A randomized comparison of indwelling pleural catheter and doxycycline pleurodesis in the management of malignant pleural effusions,” Cancer, 1999, Vol. 86, pp. 1992-1999) discloses the use of chronic indwelling pleural catheters as an effective treatment for the management of patients with symptomatic, recurrent, malignant pleural effusions.

All references are incorporated herein by their entirety.

### SUMMARY OF THE INVENTION

The present invention provides a method of producing pleurodesis in a mammalian subject, comprising administering to a subject in need thereof silver or a salt of silver in a total dosage of about 5 mg to about 500 mg over a time period of administration.

The present invention also provides a method of producing pleurodesis in a mammalian subject, comprising administering to a subject in need thereof silver or a salt of silver in an average daily dosage of about 0.005 mg/kg/day to about 2 mg/kg/day over a time period of administration.

In preferred embodiments, the time period of administration is 2 to 30 days. In some embodiments, the silver or salt of silver is administered multiple times daily, preferably two or more times daily. In some embodiments, the silver or salt of silver is administered as a continuous infusion.

The present invention also provides a kit comprising: a device for removing fluid from the pleural space of a subject, and instructions for use.

Other novel features and advantages of the present invention will become apparent to those skilled in the art upon examination of the following or upon learning by practice of the invention.

### DETAILED DESCRIPTION OF THE INVENTION

The present invention provides a method of producing pleurodesis in a mammalian subject, comprising administering to a subject in need thereof silver or a salt of silver in a total dosage of about 5 mg to about 500 mg, preferably 10 mg to about 450 mg, and more preferably about 10 mg to about 300 mg over a time period of administration. The total dosage refers to the total amount of silver of salt of silver administered over the time period of administration. The time period of administration refers to the period of time in which the silver or salt of silver is administered to the subject.

The present invention also provides a method of producing pleurodesis in a mammalian subject, comprising administering an average daily dosage of about 0.005 mg/kg/day to about 2 mg/kg/day, preferably about 0.01 mg/kg/day to about 1 mg/kg/day, more preferably about 0.05 mg/kg/day to about 0.7 mg/kg/day, over a time period of administration. The daily dosage is the amount of silver or salt of silver nitrate which is administered in a day (24 hour period). The average daily dosage is the average of the daily dosages over the time period of administration.

In some embodiments, the time period of administration is about 2 to 30 days, preferably about 3 to 21 days, more preferably about 3 to 10 days. However, if effective pleurodesis is not achieved in that time period, the silver or salt or silver may be administered for a longer period of time.

In some embodiments, the silver or salt of silver is administered two or more times daily. In some embodiments, the silver or salt of silver is administered as a continuous infusion. The rate of the continuous infusion may be the same or different over the time period of administration.

In some embodiments wherein the time period of administration exceeds 10 days, about 20% to about 95%, preferably about 30% to about 90%, and more preferably about 50% to about 85%, of the total dosage is administered over a period of 3 to 10 days.

The present invention involves administration of silver or a salt of silver. Salts of silver include, but are not limited to, nitrate, oxide, chloride, iodide, bromide, sulphide, cyanide, hyposulfite, sulfate, and nitrite. In preferred embodiments, the present invention involves administration of silver nitrate. The silver or a salt of silver used in the methods of the presently claimed invention is preferably a known sclerosing agent with a history of use in the pleural space of a mammal, preferably a human.

Pleurodesis refers to the creation of fibrous adhesion between the parietal and visceral layers of the pleura. General pleurodesis or diffuse pleurodesis refer to pleurodesis that is distributed throughout the pleural layers and not confined to a specific location. General or diffuse pleurodesis comprise adhesion or fusion of the parietal and visceral layers that is not limited to the location at which the silver nitrate is introduced into the pleural space. In preferred embodiments, in a Pleurodesis Score scale measured between 1 to 8, wherein 1 represent no adhesions and 8 multiple adhesions between the visceral and parietal pleura with symphysis involving greater than 50% of the hemithorax, pleurodesis is produced with a pleurodesis score greater than 1, preferably greater than 3, more preferably greater than 6, and most preferably 8.

In preferred embodiments of a method of producing pleurodesis, the pleurodesis is achieved with little or no pain. Pain can be measured in a variety of ways, by any method known in the art. For example, pain can potentially be measured by a pain score (0-10), using such scales as a Numerical Ratings Scale (NRS) or Visual Analog Scale (VAS), by the amount or frequency of drugs necessary to control the pain, or by the length of hospital stay that results from the pain/fever/inflammation. In some embodiments, the pleurodesis achieved by the presently claimed method would reduce pain to a level that would not require inpatient care or ≦4 on NRS).

The methods of the present invention involve administration of silver or a salt of silver to mammalian subjects. Preferably, the mammalian subjects are selected from the group consisting of: humans, sheep, dogs, cats, cows, and horses. Preferably the mammalian subject is a human.

The administration of silver or a salt of silver, preferably silver nitrate, can be completed by any known method in the art, such as those described in WO 2009/060322 and U.S. Pat. No. 6,287,285, which are each incorporated by reference. Preferably, the silver or a salt of silver is administered by catheter, wherein a catheter coated with silver or a salt of silver elutes the silver or a salt of silver. Typically, a catheter coated with silver or a salt of silver is inserted into the pleural space, where the silver or salt of silver is released and produces pleurodesis. In some embodiments, the silver or a salt of silver may be released in a sustained-release manner over a period of time to achieve a general or diffuse pleurodesis of the pleural layers. In some embodiments, greater than 50%, preferably greater than 75%, or more preferably greater than 85%, of the silver or a salt of silver is released at a decreasing rate over a period of time, preferably over 24 hours, more preferably over 72 hours, most preferably over 120 hours.

In some embodiments, administration of the silver or a salt of silver is accompanied by drainage of fluid and/or air from the pleural space. Examples of devices that assist in the drainage of fluid and/or air from the pleural space include, but are not limited to catheters, chest tubes, syringes, needles, or vacuum-assisted devices such as siphon, vacuum bottle, wall suction, or other means. Preferably the fluid is removed by a catheter, more preferably a pleural catheter. In preferred embodiments, the treatments are conducted on an outpatient basis, in patients having an indwelling pleural catheter, such as the PLEURX® Pleural Catheter, marketed by Carefusion. Putnam et al. “Outpatient Management of Malignant Pleural Effusion by a Chronic Indwelling Pleural Catheter,” Ann Thorac Surg 2000; 69:369-375. Warren et al. “Identification of clinical factors predicting PleurX® catheter removal in patients treated for malignant pleural effusion.” European Journal of Cardio-Thoracic Surgery 2008; 33(1): 89-94.

The present invention also provides a kit comprising: a device for removing fluid from the pleural space of a subject, and instructions for use. The device may be any device that assists in the drainage of fluid and/or air from the pleural space, as described above. The instructions for use comprise instructions for the method of treating pleural effusion, as described above.

The present invention also provides a method of treating, preventing, or reducing the occurrence of pleural conditions selected from the group consisting of malignant pleural effusions, benign pleural effusions, and pneumothorax.

1. A method of producing pleurodesis in a mammalian subject, comprising administering to the subject in need thereof silver or a salt of silver in a total dosage of about 5 to about 500 mg over a time period of administration.

2. The method of claim 1, wherein the total dosage is about 10 to about 450 mg.

3. The method of claim 1, wherein the total dosage is about 10 mg to about 300 mg.

4. The method of claim 1, wherein the time period of administration is about 2 to 30 days.

5. The method of claim 1, wherein the time period of administration is about 3 to 10 days.

6. A method of producing pleurodesis in a mammalian subject, comprising administering to a subject in need thereof silver or a salt of silver in an average daily dosage of about 0.005 mg/kg/day to about 2 mg/kg/day over a time period of administration.

7. The method of claim 6, wherein the average daily dosage is about 0.01 mg/kg/day to about 1 mg/kg/day.

8. The method of claim 6, wherein the average daily dosage is about 0.05 mg/kg/day to about 0.7 mg/kg/day.

9. The method of claim 6, wherein the time period of administration is about 2 to 30 days.

10. The method of claim 6, wherein the time period of administration is about 3 to 10 days.

11. The method of claim 1, wherein the mammalian subject is a human.

12. The method of claim 1, wherein the method is effective in treating or preventing pleural conditions selected from the group consisting of malignant pleural effusions, benign pleural effusions, and pneumothorax.

13. A kit comprising: a device for removing fluid from the pleural space of a mammalian subject, and instructions for use.

14. The kit of claim 13, wherein the device is selected from the group consisting of: catheter, chest tube, syringe, and needle.

15. The kit of claim 13, wherein the instructions for use comprise instructions to administer to a subject in need thereof silver or a salt of silver in a total dosage of about 5 to about 500 mg over a time period of administration.

16. The kit of claim 15, wherein the time period of administration is about 2 to about 30 days.

17. The kit of claim 13, wherein the instructions for use comprise instructions to administer to a subject in need thereof silver or a salt of silver in an average daily dosage of about 0.005 mg/kg/day to about 2 mg/kg/day over a time period of administration.

18. The kit of claim 17, wherein the time period of administration is about 2 to about 30 days.
